(19) United States
(12) Patent Application Publication
(10) Pub. No.: US 2017/0371408 A1
(43) Pub. Date: Dec. 28, 2017

(54) VIDEO DISPLAY DEVICE SYSTEM, HEARTBEAT SPECIFYING METHOD, HEARTBEAT SPECIFYING PROGRAM

(71) Applicant: FOVE, INC., San Mateo, CA (US)

(72) Inventor: Lochlainn Wilson, Tokyo (JP)

(21) Appl. No.: 15/634,777

(22) Filed: Jun. 27, 2017

(30) Foreign Application Priority Data
Jun. 28, 2016 (JP) 2016-127747

(51) Int. Cl. G06F 3/01; A61B 5/024; A61B 5/00; G06F 1/16

(52) U.S. Cl. CPC G06F 3/013; G06F 3/011; G06F 1/163; A61B 5/02438; A61B 5/7278; A61B 2562/0219

(57) ABSTRACT

A video display system capable of detecting heartbeat of a user by being mounted on the head of a user when used is provided. The video display system comprises a video display device that is mounted on the head of a user when used and a heartbeat detection device that detects the heartbeat of the user is provided, wherein video display device includes an acceleration sensor that sequentially outputs measured acceleration information and a first transmission unit that sequentially transmits the acceleration information to the heartbeat detection device, and the heartbeat detection device includes a first reception unit that receives the acceleration information transmitted from the video display device and a heartbeat detection unit that detects the heartbeat of the user from a waveform indicating a change in acceleration based on the received acceleration information.

### BACKGROUND OF THE INVENTION

### Field of the Invention

The present invention relates to a heartbeat detection technology using a video display device that is mounted on the head of a user when used.

### Description of Related Art

Conventionally, a head mounted display includes an acceleration sensor, detects an inclination and posture of a user, and displays a video in a direction the user is facing depending on the detected posture. Japanese Unexamined Patent Application Publication No. 2015-28654 discloses a head mounted display device that detects motion or a direction of a user by using an acceleration sensor or a gyro and displays a video in a gaze direction of the user.

### SUMMARY OF THE INVENTION

However, there is a demand for displaying a video with higher usability in a video display device that is mounted on the head of a user when used such as the head mounted display described in Japanese Unexamined Patent Application Publication No. 2015-28654.

The present invention has been made in consideration of the above demand, and an object thereof is to provide a video display system capable of displaying a video with higher usability.

According to an aspect of the present invention, a video display system is a video display system including a video display device that is mounted on the head of a user when used and a heartbeat detection device that detects the heartbeat of the user, wherein the video display device includes an acceleration sensor that sequentially outputs measured acceleration information and a first transmission unit that sequentially transmits the acceleration information to the heartbeat detection device, and the heartbeat detection device includes a first reception unit that receives the acceleration information transmitted from the video display device and a heartbeat detection unit that detects the heartbeat of the user from a waveform indicating a change in acceleration based on the received acceleration information.

Further, the video display system may further include a gaze detection device. The video display device may further include a display unit that displays an image and an imaging unit that captures an image of an eye of a user who views the image and is irradiated with invisible light. The first transmission unit may also transmit a captured image captured by the imaging unit to the gaze detection device. The gaze detection device may include a second reception unit that receives the captured image, a gaze detection unit that detects a gaze direction of the user via the captured image, and an image generation unit that generates an image to be displayed on the video display device on the basis of the gaze direction of the user.

Further, in the video display system, the first transmission unit may also transmit acceleration information to the gaze detection device, and the image generation unit may specify a direction of the user's body on the basis of the acceleration information and generate an image according to the specified direction.

Further, in the video display system, the heartbeat detection device may further include a second transmission unit that transmits information on the heartbeat of the user detected by the heartbeat detection unit to the gaze detection device, the second reception unit may also receive information on the heartbeat, and the image generation unit may generate an image on the basis of the information on the heartbeat.

Further, in the video display system, the heartbeat detection device may further include a storage unit that stores waveform information indicating a typical waveform of a heartbeat, and the heartbeat detection unit may detect the heartbeat of the user on the basis of a correlation between a waveform based on a change in acceleration based on the acceleration information and the waveform information.

Further, in the video display system, the heartbeat detection unit may detect the heartbeat of the user on the basis of a correlation between a waveform in a predetermined first period and another waveform in a second period included in the waveform based on the change in acceleration based on the acceleration information and a waveform.

Further, in the video display system, the video display device may be a head mounted display.

According to an aspect of the present invention, a heartbeat specifying method includes an acquiring step of sequentially acquiring acceleration information from an acceleration sensor included in a video display device that is mounted on the head of a user when used, and a specifying step of specifying the heartbeat of the user on the basis of the sequentially acquired acceleration information.

According to an aspect of the present invention, a heartbeat specifying program allows a computer to execute an acquiring function for sequentially acquiring acceleration information from an acceleration sensor included in a video display device that is mounted on the head of a user when used, and a specifying function for specifying the heartbeat of the user on the basis of the sequentially acquired acceleration information.

According to the present invention, a video display system can specify the heartbeat of a user by using an acceleration sensor that is used to specify an inclination or a direction of the user's body. Therefore, on the basis of the heartbeat, because it is possible to display a video suitable for the user by, for example, understanding the user's mental state, a video display system with high usability can be provided.

### BRIEF DESCRIPTION OF THE DRAWINGS

FIG. 1 is an external view illustrating a state in which a user wears a head mounted display according to an embodiment;

FIG. 2 is a perspective view schematically illustrating an overview of an image display system of the head mounted display according to the embodiment;

FIG. 3 is a diagram schematically illustrating an optical configuration of an image display system of the head mounted display according to the embodiment;

FIG. 4 is a block diagram illustrating a configuration of head mounted display system according to the embodiment;

FIG. 5 is a schematic diagram illustrating calibration for detection of a gaze direction according to the embodiment;

FIG. 6 is a schematic diagram illustrating position coordinates of a cornea of a user;

FIG. 7 is a flowchart illustrating an operation of the head mounted display system according to the embodiment;

FIG. 8 is a graph illustrating an example of values measured by an acceleration sensor according to the embodiment; and

FIG. 9 is a block diagram illustrating a circuit configuration of the head mounted display system.

### DETAILED DESCRIPTION OF THE INVENTION

Hereinafter, a head mounted display system 1 according to an aspect of a video display system of the present invention will be described with reference to the drawings.

### Embodiment

### <Configuration>

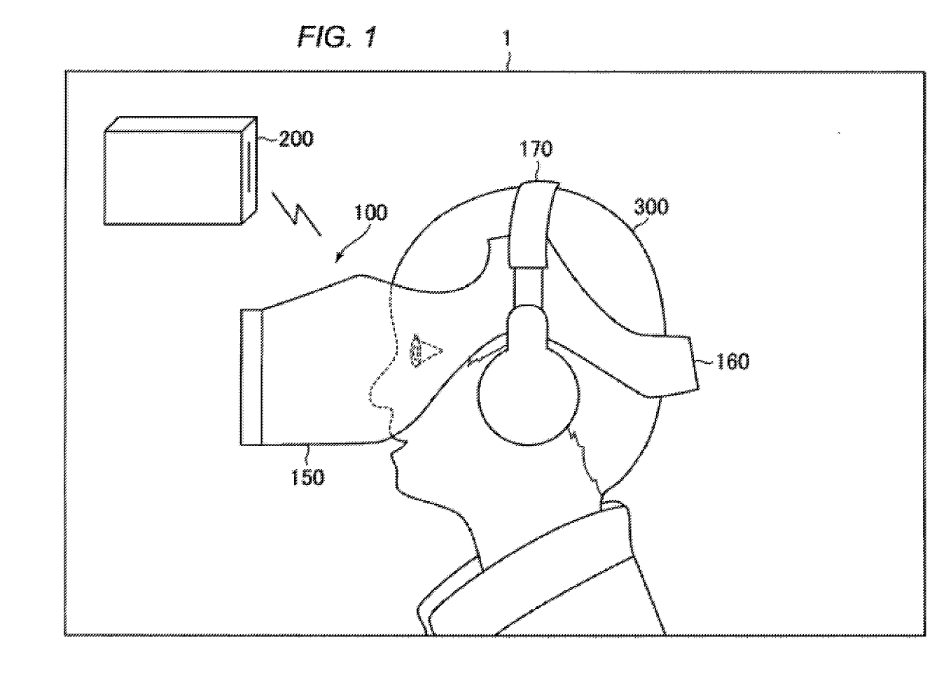

FIG. 1 is a view schematically illustrating an overview of the head mounted display system 1 according to an embodiment. The head mounted display system 1 according to the embodiment includes a head mounted display 100 and a gaze detection device 200. As illustrated in FIG. 1, the head mounted display 100 is mounted on the head of a user 300 for use.

The gaze detection device 200 detects a gaze direction of at least one of a right eye and a left eye of the user wearing the head mounted display 100 and specifies the user's focal point, i.e., a point gazed by the user in a three-dimensional image displayed on the head mounted display. The gaze detection device 200 also functions as a video generation device that generates a video to be displayed by the head mounted display 100. The gaze detection device 200 also functions as a heartbeat detection device that detects heartbeat of the user. A heartbeat detection device may be incorporated in the head mounted display system 1 as a separate device from the gaze detection device 200. In this case, the heartbeat detection device may be configured to allow a gaze detection device and a head mounted display to communicate with each other and realize heartbeat detection which will be described below. For example, the gaze detection device 200 is a device capable of reproducing videos of stationary game machines, portable game machines, PCs, tablets, smartphones, phablets, video players, TVs, or the like, but the present invention is not limited thereto. The gaze detection device 200 is wirelessly or wiredly connected to the head mounted display 100. In the example illustrated in FIG. 1, the gaze detection device 200 is wirelessly connected to the head mounted display 100. The wireless connection between the gaze detection device 200 and the head mounted display 100 can be realized using a known wireless communication technique such as Wi-Fi (registered trademark) or Bluetooth (registered trademark). For example, transfer of videos between the head mounted display 100 and the gaze detection device 200 is executed according to a standard such as Miracast (registered trademark), WiGig (registered trademark), or WHDI (registered trademark).

FIG. 1 illustrates an example in which the head mounted display 100 and the gaze detection device 200 are different devices. However, the gaze detection device 200 may be built into the head mounted display 100.

The head mounted display 100 includes a housing 150, a fitting harness 160, and headphones 170. The housing 150 houses an image display system, such as an image display element, for presenting videos to the user 300, and a wireless transfer module (not illustrated) such as a Wi-Fi module or a Bluetooth (registered trademark) module. The fitting harness 160 is used to mount the head mounted display 100 on the head of the user 300. The fitting harness 160 may be realized by, for example, a belt or an elastic band. When the user 300 wears the head mounted display 100 using the fitting harness 160, the housing 150 is arranged at a position where the eyes of the user 300 are covered. Thus, if the user 300 wears the head mounted display 100, a field of view of the user 300 is covered by the housing 150.

The headphones 170 output audio for the video that is reproduced by the gaze detection device 200. The headphones 170 may not be fixed to the head mounted display 100. Even when the user 300 wears the head mounted display 100 using the fitting harness 160, the user 300 may freely attach or detach the headphones 170.

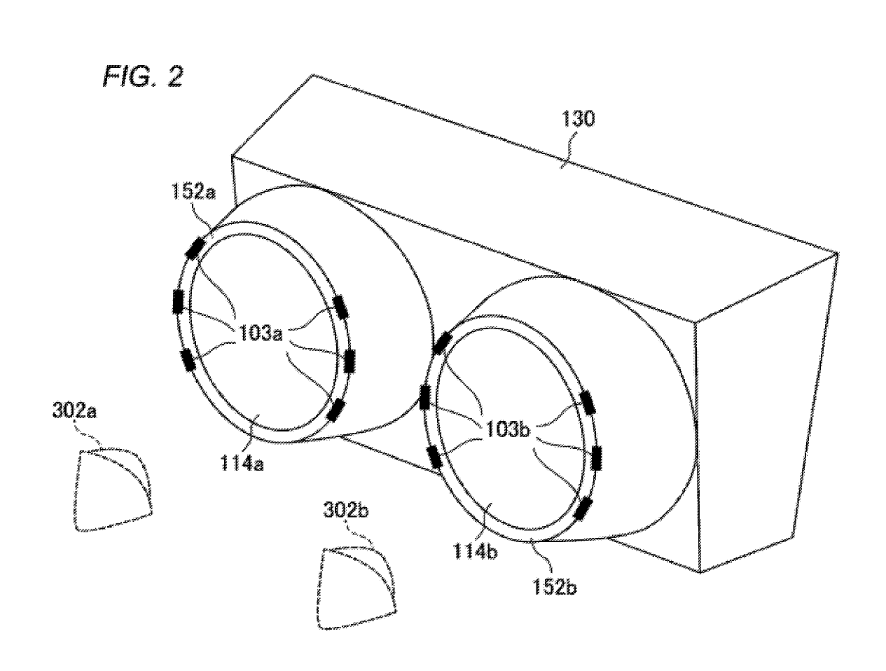

FIG. 2 is a perspective diagram illustrating an overview of the image display system 130 of the head mounted display 100 according to the embodiment. Specifically, FIG. 2 illustrates a region of the housing 150 according to an embodiment that faces corneas 302 of the user 300 when the user 300 wears the head mounted display 100.

As illustrated in FIG. 2, a convex lens 114a for the left eye is arranged at a position facing the cornea 302a of the left eye of the user 300 when the user 300 wears the head mounted display 100. Similarly, a convex lens 114b for a right eye is arranged at a position facing the cornea 302b of the right eye of the user 300 when the user 300 wears the head mounted display 100. The convex lens 114a for the left eye and the convex lens 114b for the right eye are gripped by a lens holder 152a for the left eye and a lens holder 152b for the right eye, respectively.

Hereinafter, in this specification, the convex lens 114a for the left eye and the convex lens 114b for the right eye are simply referred to as a “convex lens 114” unless the two lenses are particularly distinguished. Similarly, the cornea 302a of the left eye of the user 300 and the cornea 302b of the right eye of the user 300 are simply referred to as a “cornea 302” unless the corneas are particularly distinguished. The lens holder 152a for the left eye and the lens holder 152b for the right eye are referred to as a “lens holder 152” unless the holders are particularly distinguished.

A plurality of infrared light sources 103 are included in the lens holders 152. For the purpose of brevity, in FIG. 2, the infrared light sources that irradiate the cornea 302a of the left eye of the user 300 with infrared light are collectively referred to as infrared light sources 103a, and the infrared light sources that irradiate the cornea 302b of the right eye of the user 300 with infrared light are collectively referred to as infrared light sources 103b. Hereinafter, the infrared light sources 103a and the infrared light sources 103b are referred to as “infrared light sources 103” unless the infrared light sources 103a and the infrared light sources 103b are particularly distinguished. In the example illustrated in FIG. 2, six infrared light sources 103a are included in the lens holder 152a for the left eye. Similarly, six infrared light sources 103b are included in the lens holder 152b for the right eye. Thus, the infrared light sources 103 are not directly arranged in the convex lenses 114, but are arranged in the lens holders 152 that grip the convex lenses 114, making the attachment of the infrared light sources 103 easier. This is because machining for attaching the infrared light sources 103 is easier than for the convex lenses 114 that are made of glass or the like since the lens holders 152 are typically made of a resin or the like.

As described above, the lens holders 152 are members that grip the convex lenses 114. Therefore, the infrared light sources 103 included in the lens holders 152 are arranged around the convex lenses 114. Although there are six infrared light sources 103 that irradiate each eye with infrared light herein, the number of the infrared light sources 103 is not limited thereto. There may be at least one light source 103 for each eye, and two or more light sources 103 are desirable.

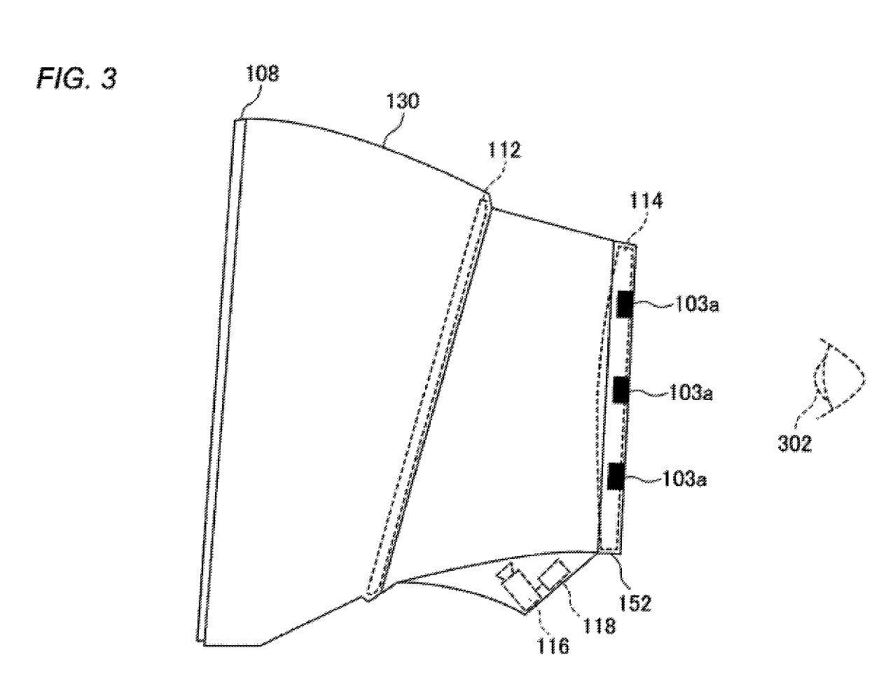

FIG. 3 is a schematic diagram of an optical configuration of the image display system 130 contained in the housing 150 according to the embodiment, and is a diagram illustrating a case in which the housing 150 illustrated in FIG. 2 is viewed from a side surface on the left eye side. The image display system 130 includes infrared light sources 103, an image display element 108, a hot mirror 112, the convex lenses 114, a camera 116, and a first communication unit 118.

The infrared light sources 103 are light sources capable of emitting light in a near-infrared wavelength region (700 nm to 2500 nm range). Near-infrared light is generally light in a wavelength region of non-visible light that cannot be observed by the naked eye of the user 300.

The image display element 108 displays an image to be presented to the user 300. The image to be displayed by the image display element 108 is generated by a video generation unit 223 in the gaze detection device 200. The video generation unit 223 will be described below. The image display element 108 can be realized by using an existing liquid crystal display (LCD), organic electro luminescence display (organic EL display), or the like.

The hot mirror 112 is arranged between the image display element 108 and the cornea 302 of the user 300 when the user 300 wears the head mounted display 100. The hot mirror 112 has a property of transmitting visible light created by the image display element 108, but reflecting near-infrared light.

The convex lenses 114 are arranged on the opposite side of the image display element 108 with respect to the hot mirror 112. In other words, the convex lenses 114 are arranged between the hot mirror 112 and the cornea 302 of the user 300 when the user 300 wears the head mounted display 100. That is, the convex lenses 114 are arranged at positions facing the corneas 302 of the user 300 when the user 300 wears the head mounted display 100.

The convex lenses 114 condense image display light that is transmitted through the hot mirror 112. Thus, the convex lenses 114 function as image magnifiers that enlarge an image created by the image display element 108 and present the image to the user 300. Although only one of each convex lens 114 is illustrated in FIG. 2 for convenience of description, the convex lenses 114 may be lens groups configured by combining various lenses or may be a plano-convex lens in which one surface has curvature and the other surface is flat.

A plurality of infrared light sources 103 are arranged around the convex lens 114. The infrared light sources 103 emit infrared light toward the cornea 302 of the user 300.

Although not illustrated in the figure, the image display system 130 of the head mounted display 100 according to the embodiment includes two image display elements 108, and can independently generate an image to be presented to the right eye of the user 300 and an image to be presented to the left eye of the user. Accordingly, the head mounted display 100 according to the embodiment may present a parallax image for the right eye and a parallax image for the left eye to the right and left eyes of the user 300. Thereby, the head mounted display 100 according to the embodiment can present a stereoscopic video that has a feeling of depth for the user 300.

As described above, the hot mirror 112 transmits visible light but reflects near-infrared light. Thus, the image light emitted by the image display element 108 is transmitted through the hot mirror 112, and reaches the cornea 302 of the user 300. The infrared light emitted from the infrared light sources 103 and reflected in a reflective area inside the convex lens 114 reaches the cornea 302 of the user 300.

The infrared light reaching the cornea 302 of the user 300 is reflected by the cornea 302 of the user 300 and is directed to the convex lens 114 again. This infrared light is transmitted through the convex lens 114 and is reflected by the hot mirror 112. The camera 116 includes a filter that blocks visible light and images the near-infrared light reflected by the hot mirror 112. That is, the camera 116 is a near-infrared camera which images the near-infrared light emitted from the infrared light sources 103 and reflected by the cornea of the eye of the user 300.

Although not illustrated in the figure, the image display system 130 of the head mounted display 100 according to the embodiment includes two cameras 116, that is, a first imaging unit that captures an image including the infrared light reflected by the right eye and a second imaging unit that captures an image including the infrared light reflected by the left eye. Thereby, images for detecting gaze directions of both the right eye and the left eye of the user 300 can be acquired.

The first communication unit 118 outputs the image captured by the camera 116 to the gaze detection device 200 that detects the gaze direction of the user 300. Specifically, the first communication unit 118 transmits the image captured by the camera 116 to the gaze detection device 200. Although the gaze detection unit 221 functioning as a gaze direction detection unit will be described below in detail, the gaze direction unit is realized by a heartbeat specifying program executed by a central processing unit (CPU) of the gaze detection device 200. When the head mounted display 100 includes computational resources such as a CPU or a memory, the CPU of the head mounted display 100 may execute the program that realizes the gaze direction detection unit.

As will be described below in detail, bright spots caused by near-infrared light reflected by the cornea 302 of the user 300 and an image of the eyes including the cornea 302 of the user 300 observed in a near-infrared wavelength region are captured in the image captured by the camera 116.

Although the configuration for presenting the image to the left eye of the user 300 in the image display system 130 according to the embodiment has mainly been described above, a configuration for presenting an image to the right eye of the user 300 is the same as above.

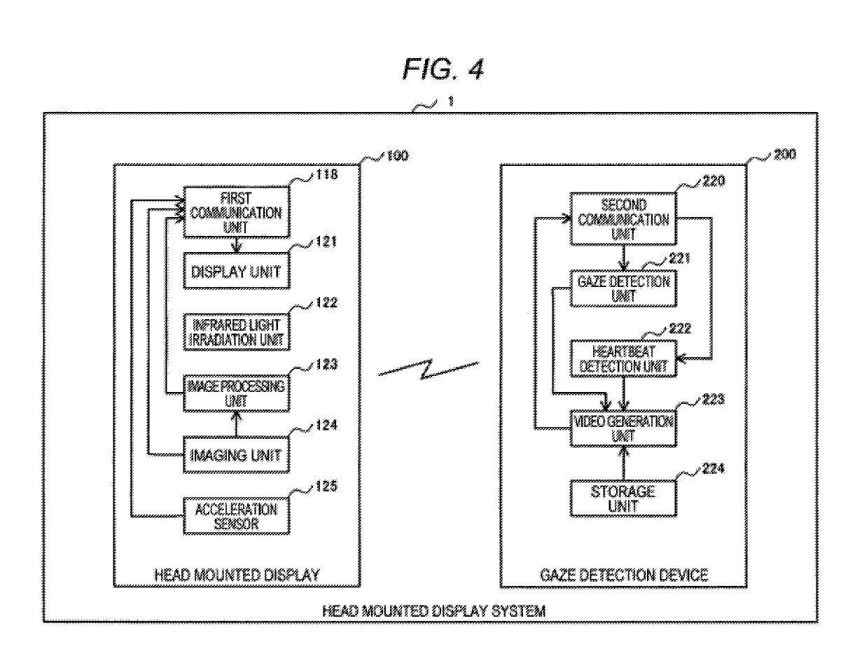

FIG. 4 is a block diagram of the head mounted display 100 and the gaze detection device 200 of the head mounted display system 1. As illustrated in FIG. 4 and as described above, the head mounted display system 1 includes the head mounted display 100 and the gaze detection device 200 that communicate with each other.

As illustrated in FIG. 4, the head mounted display 100 includes the first communication unit 118, a display unit 121, an infrared light irradiation unit 122, an image processing unit 123, an imaging unit 124, and an acceleration sensor 125.

The first communication unit 118 is a communication interface having a function of communicating with the second communication unit 220 of the gaze detection device 200. As described above, the first communication unit 118 communicates with the second communication unit 220 through wired or wireless communication. Examples of usable communication standards are as described above. The first communication unit 118 transmits image data to be used for gaze detection transferred from the imaging unit 124 or the image processing unit 123 to the second communication unit 220. The first communication unit 118 transmits acceleration information transferred from the acceleration sensor 125 to the second communication unit 220. Further, the first communication unit 118 transfers image data or a marker image transmitted from the gaze detection device 200 to the display unit 121. The image data may be a pair of parallax images including a parallax image for the right eye and a parallax image for the left eye for displaying a three-dimensional image.

The display unit 121 has a function of displaying image data transferred from the first communication unit 118 to the image display element 118. Further, the display unit 121 displays the marker image output from the video generation unit 223 on designated coordinates of the image display element 108. Further, the display unit 121 specifies the user's posture (direction) on the basis of the acceleration information transferred from the acceleration sensor 125 and displays a video in the specified direction on the image display element 108.

The infrared light irradiation unit 122 controls the infrared light sources 103 and irradiates the right eye or the left eye of the user with infrared light.

The image processing unit 123 performs image processing on the image captured by the imaging unit 124 as necessary, and transfers a processed image to the first communication unit 118.

The imaging unit 124 uses the camera 116 to capture an image including near-infrared light reflected from each eye. That is, the camera 116 performs imaging based on invisible light. Further, the imaging unit 124 captures an image including the user's eye viewing the marker image displayed on the image display element 108. The imaging unit 124 transfers the image obtained by capturing to the first communication unit 118 or the image processing unit 123 in association with a capturing time at which the image is captured.

The acceleration sensor 125 is a sensor included in the head mounted display 100 to detect acceleration. The acceleration sensor 125 transfers the detected acceleration to the first communication unit 118 and the display unit 121. The acceleration sensor 125 obtains information on acceleration of a three-axis component. A three-axis acceleration component is, for example, a vertical component and two-axis components that are orthogonal to an axis detecting the vertical orthogonal component and are orthogonal to each other.

The configuration of the head mounted display 100 has been described above.

As illustrated in FIG. 4, the gaze detection device 200 includes the second communication unit 220, the gaze detection unit 221, a heartbeat detection unit 222, the video generation unit 223, and a storage unit 224.

The second communication unit 220 is a communication interface having a function of communicating with the first communication unit 118 of the head mounted display 100. As described above, the second communication unit 220 communicates with the first communication unit 118 through wired communication or wireless communication. The second communication unit 220 transmits the image data for displaying the virtual space image transferred from the image generation unit 223, the marker image used for the calibration, and the like to the head mounted display 100. Further, the second communication unit 220 transfers an image including the user's eye viewing the marker image captured by the imaging unit 124 transferred from the head mounted display 100 or an image including the user's eye viewing an image displayed on the basis of the image data output by the video generation unit 223 to the gaze detection unit 221. Further, the second communication unit 220 transfers the acceleration information transferred from the head mounted display 100 to the heartbeat detection unit 222.

The gaze detection unit 221 receives the image data for detecting a gaze of the right eye of the user from the second communication unit 220 and detects a gaze direction of the user's right eye. The gaze detection unit 221 calculates a right-eye gaze vector indicating the gaze direction of the right eye of the user by using a method which will be described below. Likewise, the gaze detection unit 221 receives the image data for detecting a gaze of the left eye of the user from the second communication unit 220 and calculates a left-eye gaze vector indicating the gaze direction of the left eye of the user 300. Then, the gaze detection unit 221 uses the calculated gaze vectors to specify a point viewed by the user in the image displayed on the image display element 108. Further, the gaze detection unit 221 transmits the calculated gaze vectors as information on gaze directions, together with imaging time information associated with the captured image used for calculating the gaze vectors, to the head mounted display 100 via the second communication unit 220 Further, the information on gaze directions may also be information on a gaze point specified by the gaze detection unit 221.

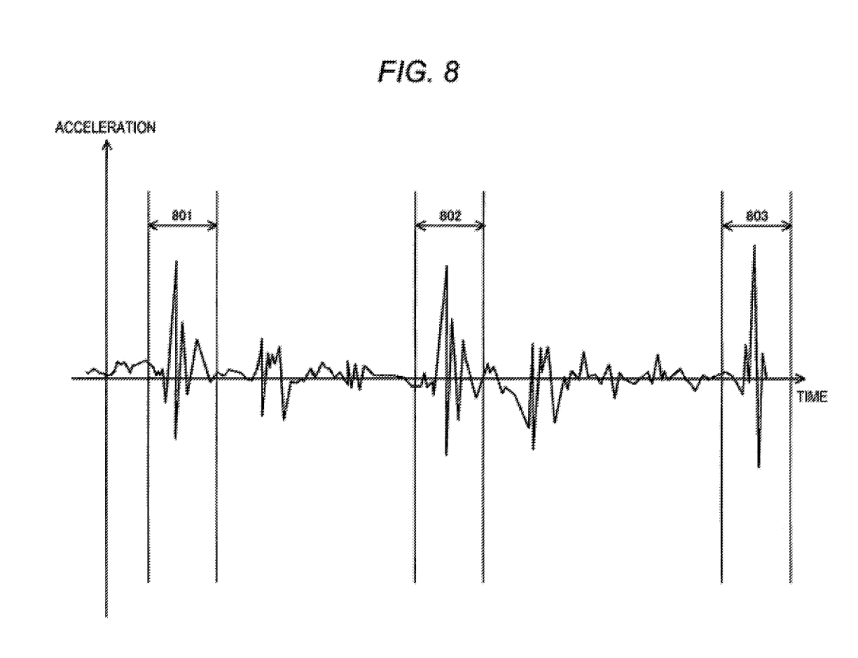

The heartbeat detection unit 222 has a function of specifying the heartbeat of the user wearing the head mounted display 100 on the basis of the acceleration information transferred from the second communication unit 220. Because the heartbeat detection unit 222 sequentially acquires acceleration information from the second communication unit 220, it is possible to obtain time-series information on the acceleration. Therefore, with respect to one axis (e.g. a vertical component) of the three-axis components of the acquired acceleration information, the heartbeat detection unit 222 plots acceleration of one axis of the acquired acceleration information on a graph in which acceleration is represented on the vertical axis and time is represented on the horizontal axis as illustrated in FIG. 8. The heartbeat detection unit 222 performs an autocorrelation with respect to the waveform of the graph obtained as above and determines whether the same waveform is periodically obtained. Specifically, the heartbeat detection unit 222 extracts a waveform having a predetermined length from the graph illustrated in FIG. 8 and stores the waveform in the storage unit 224. Then, the extracted waveform stored in the storage unit 224 is shifted in the time axis direction with respect to the original waveform, and an autocorrelation is performed. Then, periodical appearances of sections where correlation values obtained from autocorrelation exceed a predetermined threshold value are detected, and a waveform portion with high correlation value is specified as the heartbeat of the user wearing the head mounted display 100. Referring to the example of FIG. 8, it is detected that correlation values between a section 801 and a section 802 are high and correlation values between the section 802 (801) and the section 803 are high, and the sections are specified as the heartbeat of the user. The heartbeat detection unit 222 transmits the detected heartbeat information to the video generation unit 223. Here, the heartbeat information includes the heart rate per unit time, the strength of the fluctuation of the heartbeat (amplitude of acceleration), and the like.

The video generation unit 223 generates image data to be displayed on the display unit 121 of the head mounted display 100 and transfers the image data to the second communication unit 220. The video generation unit 223 generates, for example, image data for displaying a virtual space image. Further, the video generation unit 223 generates a marker image for calibration for gaze detection, and transfers the marker image together with positions of display coordinates thereof to the second communication unit 220 to transmit the marker image to the head mounted display 100. When displaying a video with a wider display range than the image display element 108 of the head mounted display 100 (e.g., a 360°-video) on the head mounted display 100, the video generation unit 223 may transfer a wide video with low resolution to the second communication unit 220, generate high-resolution image data of an image in a predetermined range including coordinates of points corresponding to the gaze directions detected by the gaze detection unit 221, and transfer the image data to the second communication unit 220. As a result, images of the video themselves can be presented to the user without pause, and a high-resolution video can be provided to the user while an amount of data sent from the gaze detection device 200 to the head mounted display 100 is suppressed. Therefore, usability can be improved. Further, the video generation unit 223 can process a video to be output on the basis of the heartbeat information transferred from the heartbeat detection unit 222. When, for example, the heartbeat of the user (heart rate per unit time) is a predetermined threshold value or higher, because there is a possibility that the user is unusually excited, the video generation unit 223 transfers a video that is processed by lowering a brightness value to the second communication unit 220 as a video to be displayed on the head mounted display 100. Also, when, for example, the heartbeat of the user is lower than the predetermined threshold value, because there is a possibility that the user is sleepy, the video generation unit 223 transfers a video that is processed by increasing the brightness value to the second communication unit 220 as a video to be displayed on the head mounted display 100. During the above processes, it is possible to perform processing with respect to gaze points of an image specified on the basis of the gaze directions detected by the gaze detection unit 221.

The storage unit 224 is a recording medium that stores various programs or data required for operation of the gaze detection device 200. The storage unit 224 is realized by, for example, a hard disk drive (HDD), a solid state drive (SSD), etc. Next, gaze direction detection according to an embodiment will be described.

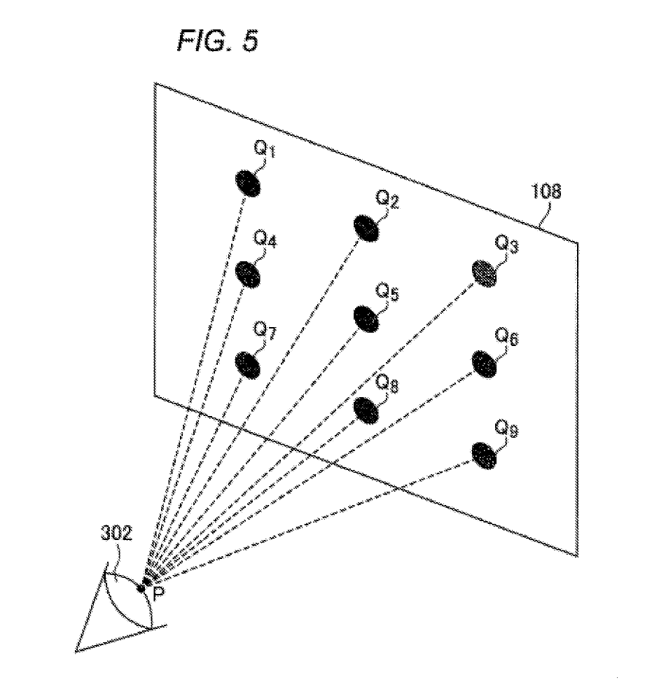

FIG. 5 is a schematic diagram illustrating calibration for detection of the gaze direction according to the embodiment. The gaze direction of the user 300 is realized by the gaze detection unit 221 in the gaze detection device 200 analyzing the video captured by the camera 116 and output to the gaze detection device 200 by the first communication unit 118.

The video generation unit 223 generates nine points (marker images) including points Q₁ to Q₉ as illustrated in FIG. 5, and causes the points to be displayed by the image display element 108 of the head mounted display 100. The gaze detection device 200 causes the user 300 to sequentially gaze at the points Q₁ up to Q₉. In this case, the user 300 is requested to gaze at each of the points by moving his or her eyeballs as much as possible without moving his or her neck. The camera 116 captures images including the cornea 302 of the user 300 when the user 300 is gazing at the nine points including the points Q₁ to Q₉.

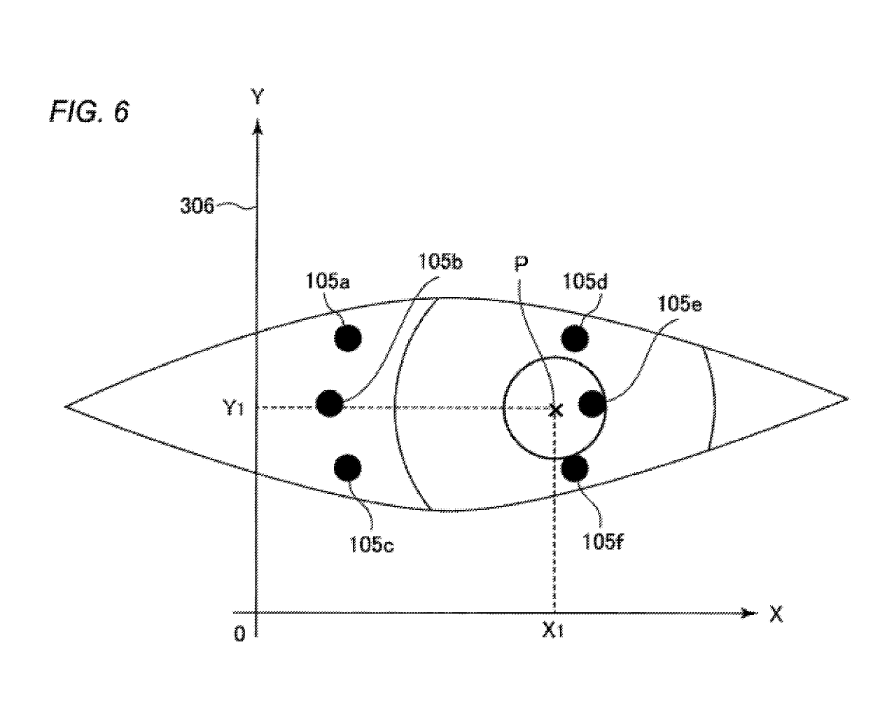

FIG. 6 is a schematic diagram illustrating the position coordinates of the cornea 302 of the user 300. The gaze detection unit 221 in the gaze detection device 200 analyzes the images captured by the camera 116 and detects bright spots 105 derived from the infrared light. When the user 300 gazes at each point by moving only his or her eyeballs, the positions of the bright spots 105 are considered to be stationary regardless of the point at which the user gazes. Thus, on the basis of the detected bright spots 105, the gaze detection unit 221 sets a two-dimensional coordinate system 306 in the image captured by the camera 116.

Further, the gaze detection unit 221 detects the center P of the cornea 302 of the user 300 by analyzing the image captured by the camera 116. This is realized by using known image processing such as the Hough transform or an edge extraction process. Accordingly, the gaze detection unit 221 can acquire the coordinates of the center P of the cornea 302 of the user 300 in the set two-dimensional coordinate system 306.

In FIG. 5, the coordinates of the points Q₁ to Q₉ in the two-dimensional coordinate system set for the display screen displayed by the image display element 108 are Q₁(x1, y1)^{T}, Q₂(x2, y2)^{T}, . . . , Q₉(x9, x9)^{T}, respectively. The coordinates are, for example, a number of a pixel located at a center of each point. Further, the center points P of the cornea 302 of the user 300 when the user 300 gazes at the points Q1 to Q9 are labeled P₁ to P₉. In this case, the coordinates of the points P1 to P9 in the two-dimensional coordinate system 306 are P₁(X1, Y1)^{T}, P₂(X2, Y2)^{T}, . . . , P₉(Z9, Y9)^{T}. T represents a transposition of a vector or a matrix.

A matrix M with a size of 2×2 is defined as Equation (1) below.

$$M = ( \begin{matrix}{m}_{11} & {m}_{12} \\ {m}_{21} & {m}_{22}\end{matrix} ) \qquad ( 1 )$$

In this case, if the matrix M satisfies Equation (2) below, the matrix M is a matrix for projecting the gaze direction of the user 300 onto an image plane that is displayed by the image display element 108.

P_{N}=MQ_{N}(N=1, . . . ,9) (2)

When Equation (2) is written specifically, Equation (3) below is obtained.

$$( \begin{matrix}{x}_{1} & {x}_{2} & … & {x}_{9} \\ {y}_{1} & {y}_{2} & … & {y}_{9}\end{matrix} ) = ( \begin{matrix}{m}_{11} & {m}_{12} \\ {m}_{21} & {m}_{22}\end{matrix} ) ( \begin{matrix}{X}_{1} & {X}_{2} & … & {X}_{9} \\ {Y}_{1} & {Y}_{2} & … & {Y}_{9}\end{matrix} ) \qquad ( 3 )$$

By transforming Equation (3), Equation (4) below is obtained.

$$( \begin{matrix}{x}_{1} \\ {x}_{2} \\ ⋮ \\ {x}_{9} \\ {y}_{1} \\ {y}_{2} \\ ⋮ \\ {y}_{9}\end{matrix} ) = ( \begin{matrix}{X}_{1} & {Y}_{1} & 0 & 0 \\ {X}_{2} & {Y}_{2} & 0 & 0 \\ ⋮ & ⋮ & ⋮ & ⋮ \\ {X}_{9} & {Y}_{9} & 0 & 0 \\ 0 & 0 & {X}_{1} & {Y}_{1} \\ 0 & 0 & {X}_{2} & {Y}_{2} \\ ⋮ & ⋮ & ⋮ & ⋮ \\ 0 & 0 & {X}_{9} & {Y}_{9}\end{matrix} ) ( \begin{matrix}{m}_{11} \\ {m}_{12} \\ {m}_{21} \\ {m}_{22}\end{matrix} ) \qquad ( 4 )$$

By the above, Equation (5) below is obtained.

y=Ax (5)

In Equation (5), elements of the vector y are known since these are coordinates of the points Q₁ to Q₉ that are displayed on the image display element 108 by the gaze detection unit 221. Further, the elements of the matrix A can be acquired since the elements are coordinates of a vertex P of the cornea 302 of the user 300. Thus, the gaze detection unit 221 can acquire the vector y and the matrix A. A vector x that is a vector in which elements of a transformation matrix M are arranged is unknown. Since the vector y and matrix A are known, an issue of estimating matrix M becomes an issue of obtaining the unknown vector x.

Equation (5) becomes the main issue to decide if the number of equations (that is, the number of points Q presented to the user 300 by the gaze detection unit 221 at the time of calibration) is larger than the number of unknown numbers (that is, the number 4 of elements of the vector x). Since the number of equations is nine in the example illustrated in Equation (5), Equation (5) is the main issue to decide.

An error vector between the vector y and the vector Ax is defined as vector e. That is, e=y−Ax. In this case, a vector xₒₚₜ that is optimal in the sense of minimizing the sum of squares of the elements of the vector e can be obtained from Equation (6) below.

xₒₚₜ=(A_{T}A)₋₁A_{T}y (6)

Here, “−1” indicates an inverse matrix.

The gaze detection unit 221 forms the matrix M of Equation (1) by using the elements of the obtained vector xₒₚₜ. Accordingly, by using coordinates of a vertex P of the cornea 302 of the user 300 and the matrix M, the gaze detection unit 221 may estimate which portion of the video displayed on the image display element 108 the right eye of the user 300 is viewing. Here, the gaze detection unit 221 also receives information on a distance between the eye of the user and the image display element 108 from the head mounted display 100 and modifies the estimated coordinate values of the gaze of the user according to the distance information. The deviation in estimation of the gaze position due to the distance between the eye of the user and the image display element 108 may be ignored as an error range. Accordingly, the gaze detection unit 221 can calculate a right gaze vector that connects a gaze point of the right eye on the image display element 108 to a vertex of the cornea of the right eye of the user. Similarly, the gaze detection unit 221 can calculate a left gaze vector that connects a gaze point of the left eye on the image display element 108 to a vertex of the cornea of the left eye of the user. A gaze point of the user on a two-dimensional plane can be specified with a gaze vector of only one eye, and information on a depth direction of the gaze point of the user can be calculated by obtaining gaze vectors of both eyes. In this manner, the gaze detection device 200 may specify a gaze point of the user. The method of specifying a gaze point described herein is merely an example, and a gaze point of the user may be specified using methods other than that according to this embodiment.

### <Operation>

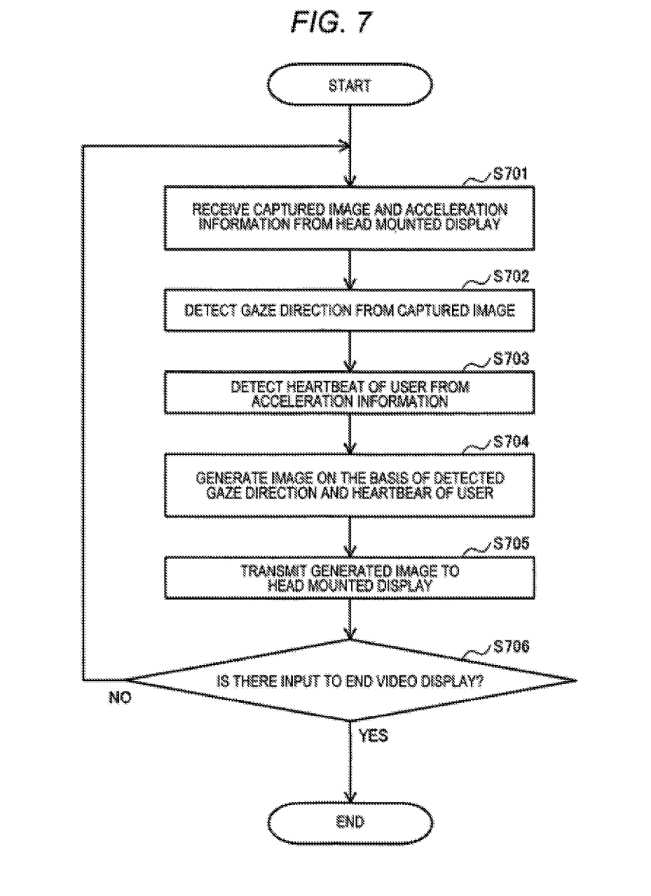

Hereinafter, the operation of the head mounted display system 1 according to the present embodiment will be described. FIG. 7 is a flowchart illustrating an operation of the head mounted display system 1 and is a flowchart illustrating a process of specifying a heartbeat on the basis of acceleration output from the acceleration sensor included in the head mounted display 100.

The second communication unit 220 of the gaze detection device 200 receives the captured image based on near-infrared light that is captured by the camera 116 and the acceleration information detected by the acceleration sensor 125 from the head mounted display 100 (step S701). The second communication unit 220 transfers the received captured image to the gaze detection unit 221 and transfers the acceleration information to the heartbeat detection unit 222.

Upon receiving the captured image, the gaze detection unit 221 uses the above method on the basis of the captured image and detects a gaze direction of the user (step S702). Further, the gaze detection unit 221 specifies a point (coordinates) in the image viewed by the user. The gaze detection unit 221 transfers the detected gaze direction and gaze point information to the video generation unit 223.

Upon receiving the acceleration information, the heartbeat detection unit 222 uses the above method and detects the heartbeat of the user from the time-series changes of acceleration components received so far which are on the same axis as a single-axis component of the acceleration information (step S702). The heartbeat detection unit 222 transfers the detected heartbeat information to the video generation unit 223.

The video generation unit 223 generates a video to be displayed on the image display element 108 of the head mounted display 100 on the basis of the transferred gaze direction information, gaze point information, and heartbeat information (step S704).

The video generation unit 223 transmits the generated video to the head mounted display 100 via the second communication unit 220 (step S705). Accordingly, an image generated on the basis of the gaze direction and the heartbeat information of the user wearing the head mounted display 100 is displayed on the image display element 108 of the head mounted display 100.

The head mounted display 100 or the gaze detection device 200 of the head mounted display system 1 determines whether an input to end the video display is received from the user (step S706). When the input to end the video display is not received (NO to step S706), the process returns to step S701. When the input to end the video display is received (YES to step S706), the process ends. The operation of the head mounted display system 1 according to the present embodiment has been described above.

### <Summary>

As described above, the head mounted display system according to the present embodiment can detect the heartbeat of the user by using the acceleration sensor used for detecting the posture or the like of the user on the head mounted display. Accordingly, it is possible to understand the state of the user viewing a video using the head mounted display, analyze the relationship between features of the video being viewed and a mental state of the user, and, on the basis of such analysis, generate and display an effective video to be viewed by the user.

### <Supplement>

The video display system according to the present invention is not limited to the above embodiment and may also be realized using other methods to realize the idea of the invention. Hereinafter, other embodiments that may be included as the idea of the present invention will be described.

(1) The hot mirror described in the above embodiment may be anything that transmits visible light and reflects invisible light, and other optical elements may be used. For example, instead of the hot mirror, an optical element such as a half mirror, a prism, an optical filter, or the like having a characteristic of transmitting visible light and reflecting invisible light may be used.

(2) Although the heartbeat detection unit 222 transmits the detected heartbeat information to the video generation unit 223 according to the above-described embodiment, the heartbeat detection unit 222 may transmit the heartbeat information to the second communication unit 220, and the heartbeat information may be transmitted to an external device from the second communication unit 220. Alternatively, the heartbeat detection unit 222 may store the heartbeat information in the storage unit 224.

(3) Although the heartbeat information detected on the basis of the acceleration information is used in processing a video by the video generation unit 223 according to the above-described embodiment, the heartbeat information may also be used to analyze data.

For example, pieces of information on whether a user is excited or bored by a certain video and, in that case, which point of the video the user is viewing may be analyzed from the acquired heartbeat information. Such an analysis allows a more effective video to be created when creating a new video.

Also, for example, from heartbeat information detected when a user plays a game using the head mounted display 100, whether a user is eased or anxious about playing the game may be analyzed. A game with higher usability may be created on the basis of such information when creating a new game.

(4) Although a heartbeat is detected on the basis of a single-axis component of acceleration components according to the above-described embodiment, a heartbeat may be detected from each of three-axis components, and a heartbeat may be specified from an average value thereof. That is, the heartbeat of the user may be specified from an average value of a period of the heartbeat obtained from a vertical component, a period of the heartbeat obtained from a single-axis component of two-axis components orthogonal to the vertical component, and a period of the heartbeat obtained from the other axis component of the two-axis components orthogonal to the vertical component.

(5) Although an example in which a heartbeat is detected by performing autocorrelation with respect to a waveform based on acceleration information has been given according to the above-described embodiment, a method of detecting a heartbeat is not limited thereto. Other methods may be used as long as the heartbeat can be detected on the basis of acceleration information. For example, a waveform sample of the heartbeat of the user may be pre-stored in the storage unit 224, and the heartbeat detection unit 222 may perform correlation with the pre-stored sample in the storage unit 224 and detect a point with high correlation values as the heartbeat of the user.

(6) In the above-described embodiment, the heartbeat detection unit 222 may further perform heart rate variation (HRV) analysis on the specified heartbeat to estimate a mental state of the user. Information indicating the estimated mental state may be output, and the video generation unit 223 may generate a video to be displayed on the head mounted display 100 on the basis of the information indicating the mental state. For example, when the user is watching a horror movie and the heartbeat is slower than a predetermined threshold value, because it can be estimated that the user is not feeling tense, the video may be processed so that a brightness difference between bright times and dark times in the video is increased to improve thrill. Also, for example, when the user is watching a suspenseful movie and the heartbeat is faster than the predetermined threshold value, it can be estimated that the user is thrilled more than necessary, and a process for delaying a frame rate compared to the previous frame rate may be performed. Also, an estimated mental state may be used in transition of video creation or game creation. For example, which kind of video or game the user is interested in may be specified on the basis of an estimated mental state and may be reflected in a video or a game being created. Also, the head mounted display system 1 may be executed until heartbeat information is output, and the HRV analysis may be executed by an external device at an output destination.

(7) Although the gaze detection device 200 includes the heartbeat detection unit 222 and detects the heartbeat of the user according to the above-described embodiment, the head mounted display 100 may include the heartbeat detection unit 222 and detect a heartbeat, or a heartbeat detection device, different from the gaze detection device 200, that includes the heartbeat detection unit 222 may detect a heartbeat. Other than the heartbeat detection unit 222, the heartbeat detection device may include at least a communication unit that receives acceleration information from the head mounted display 100, and the communication unit may transmit heartbeat information detected by the heartbeat detection unit to the gaze detection device 200.

(8) The method related to gaze detection in the above-described embodiment is merely an example, and a method of detecting a gaze using the head mounted display 100 and the gaze detection device 200 is not limited thereto.

First, although an example in which a plurality of infrared light sources that radiate near-infrared light as invisible light are provided is given in the above-described embodiment, a method or irradiating an eye of a user with near-infrared light is not limited thereto. For example, each pixel that constitutes the image display element 108 of the head mounted display 100 may include sub-pixels that emit near-infrared light, and the sub-pixels that emit near-infrared light may be caused to selectively emit light to irradiate an eye of a user with near-infrared light. Alternatively, the head mounted display 100 include a retinal projection display instead of the image display element 108 and realize near-infrared irradiation by displaying using the retinal projection display and including pixels that emit a near-infrared light color in the image projected to the retina of the user. Sub-pixels that emit near-infrared light may be regularly changed for both the image display element 108 and the retinal projection display. The hot mirror 112 according to the above-described embodiment is unnecessary in the case in which sub-pixels that emit near-infrared light are provided as sub-pixels in the image display element 108 or the case in which pixels of near-infrared light are included in the retinal projection display.

Further, the gaze detection algorithm given in the above-described embodiment is not limited to the method given in the above-described embodiment, and other algorithms may be used as long as gaze detection can be realized.

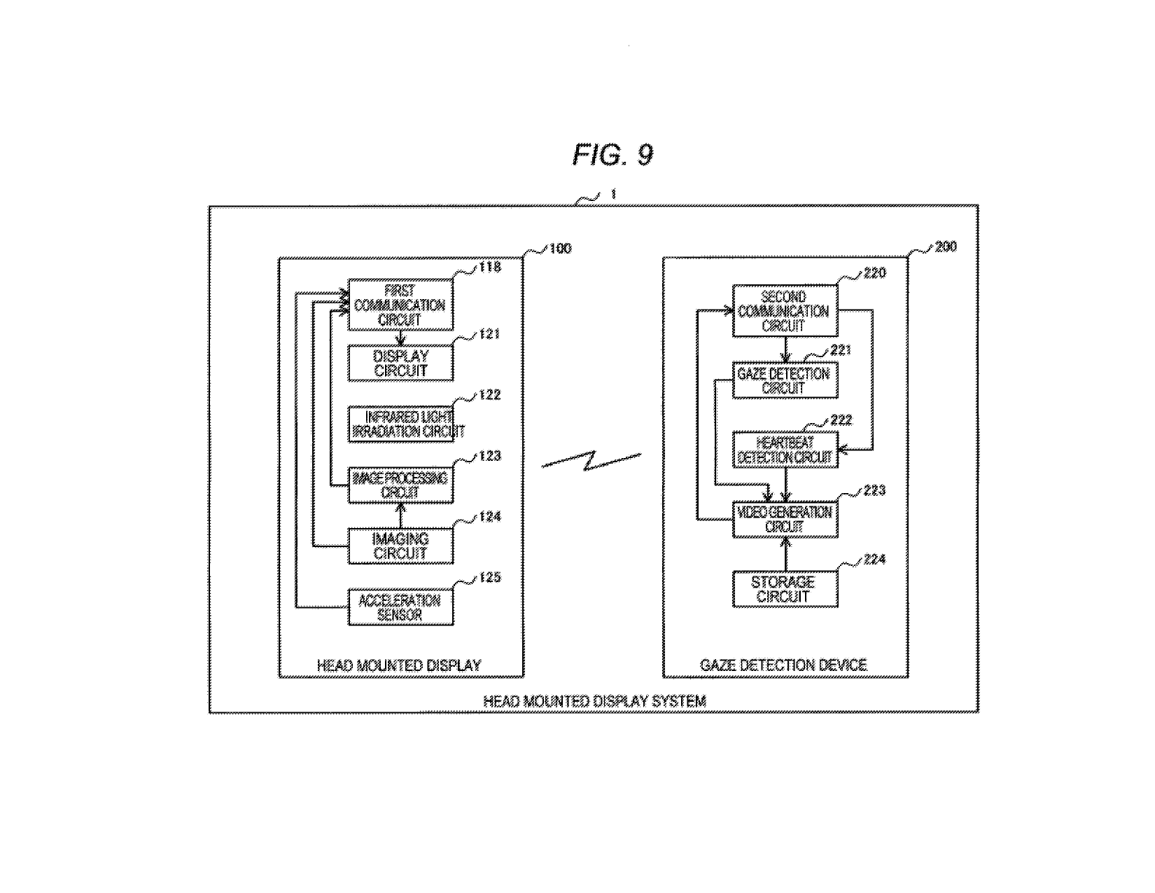

(9) Although a processor of the gaze detection device 200 specifies the heartbeat of the user wearing the head mounted display 100 by executing a heartbeat specifying program or the like according to the above-described embodiment, the heartbeat of the user may also be specified by a logic circuit (hardware) or a dedicated circuit formed in an integrated circuit (IC) chip, a large scale integration (LSI), or the like of the gaze detection device 200. These circuits may be realized by one or a plurality of ICs, and functions of a plurality of functional parts in the above-described embodiment may be realized by a single IC. The LSI is sometimes referred to as VLSI, super LSI, ultra LSI, etc. due to the difference in integration degree. That is, as illustrated in FIG. 9, the head mounted display 100 may include a first communication unit 118a, a first display circuit 121a, an infrared light irradiation circuit 122a, an image processing circuit 123a, an imaging circuit 124a, and the acceleration sensor 125, and functions thereof are the same as those of respective parts with the same names given in the above-described embodiment. Further, the gaze detection device 200 may include a second communication circuit 220a, a gaze detection circuit 221a, a heartbeat detection circuit 222a, a video generation circuit 223a, and a storage circuit 224a, and functions thereof are the same as those of respective parts with the same names given in the above-described embodiment.

The heartbeat specifying program may be recorded in a processor-readable recording medium, and a “non-transient tangible medium” such as a tape, a disc, a card, a semiconductor memory, and a programmable logic circuit may be used as the recording medium. Further, the heartbeat specifying program may be supplied to the processor via any transmittable transmission medium (a communication network, broadcast waves, or the like). The present invention can also be realized in the form of a data signal embedded in carrier waves in which the heartbeat specifying program is implemented by electronic transmission.

The heartbeat specifying program may be implemented using, for example, a script language such as ActionScript, JavaScript (registered trademark), Python, or Ruby and a compiler language such as C language, C++, C#, Objective-C, or Java (registered trademark).

(10) The configurations given in the above-described embodiment and each (supplement) may be appropriately combined.

(11) In the head mounted display system according to an aspect of the video display system of the above-described embodiment, the head mounted display 100 may be any device other than a head mounted display as long as the device is mounted on the head of a user when used, displays a video, and includes an acceleration sensor. A device other than a head-mounted display, e.g., glasses, may be used instead. In this case, the glasses should include an acceleration sensor and functions of the head mounted display 100 such as a function of displaying a video on a glass portion of the glasses, a function of capturing an image of a user's eyes, and a function of irradiating a user's eye with near-infrared light.

1. A video display system comprising a video display device that is mounted on the head of a user when used and a heartbeat detection device that detects the heartbeat of the user,
wherein the video display device includes
an acceleration sensor that sequentially outputs measured acceleration information; and
a first transmission unit that sequentially transmits the acceleration information to the heartbeat detection device, and
the heartbeat detection device includes
a first reception unit that receives the acceleration information transmitted from the video display device; and
a heartbeat detection unit that detects the heartbeat of the user from a waveform indicating a change in acceleration based on the received acceleration information.

2. The video display system according to claim 1, further comprising a gaze detection device,
wherein the video display device further includes
a display unit that displays an image; and
an imaging unit that captures an image of an eye of a user that views the image and is irradiated with invisible light,
the first transmission unit transmits a captured image captured by the imaging unit to the gaze detection device, and
the gaze detection device includes
a second reception unit that receives the captured image;
a gaze detection unit that detects a gaze direction of the user via the captured image; and
an image generation unit that generates an image to be displayed on the video display device on the basis of the gaze direction of the user.

3. The video display system according to claim 2,
wherein the first transmission unit transmits acceleration information to the gaze detection device, and
the image generation unit specifies a direction of the user's body on the basis of the acceleration information and generates an image according to the specified direction.

4. The video display system according to claim 2,
wherein the heartbeat detection device further includes a second transmission unit that transmits information on the heartbeat of the user detected by the heartbeat detection unit to the gaze detection device,
the second reception unit receives information on the heartbeat, and
the image generation unit generates an image on the basis of the information on the heartbeat.

5. The video display system according to claim 1,
wherein the heartbeat detection device further includes a storage unit that stores waveform information indicating a typical waveform of a heartbeat, and
the heartbeat detection unit detects the heartbeat of the user on the basis of a correlation between a waveform based on a change in acceleration based on the acceleration information and the waveform information.

6. The video display system according to claim 1,
wherein the heartbeat detection unit detects the heartbeat of the user on the basis of a correlation between a waveform in a predetermined first period and another waveform in a second period included in the waveform based on the change in acceleration based on the acceleration information.

7. The video display system according to claim 1,
wherein the video display device is a head mounted display.

8. A heartbeat specifying method comprising
an acquiring step of sequentially acquiring acceleration information from an acceleration sensor included in a video display device that is mounted on the head of a user when used, and
a specifying step of specifying the heartbeat of the user on the basis of the sequentially acquired acceleration information.

9. A heartbeat specifying program that allows a computer to execute
an acquiring function for sequentially acquiring acceleration information from an acceleration sensor included in a video display device that is mounted on the head of a user when used, and
a specifying function for specifying the heartbeat of the user on the basis of the sequentially acquired acceleration information.
